(19) United States
(12) Patent Application Publication
(10) Pub. No.: US 2007/0021740 A1
(43) Pub. Date: Jan. 25, 2007

(54) Laser Treatment of Hypertrophic Obstructive Cardiomyopathy (HOCM)

(75) Inventor: Helmut Weber, Munich (DE)

Correspondence Address: HELMUT WEBER, MD, ZENNERSTR. 16, MUNICH, D-81379, DE

(73) Assignee: LASCOR GMBH, Munich (DE)

(21) Appl. No.: 11/161,125

(22) Filed: Jul. 24, 2005

(51) Int. Cl. A61B 18/20; A61B 19/00

(52) U.S. Cl. 606/15; 606/10; 128/898

(57) ABSTRACT

The invention relates to a method for treatment of Hypertrophic Obstructive Cardiomyopathy (HOCM) in a human heart, wherein a laser catheter means is introduced into a patient's body and advanced into a heart cavity and directed towards a hypertrophied region of the heart. Ventricular outflow tract obstructions may be substantially reduced or even abolished when laser light is applied to the hypertrophied tissue so as to produce a coagulation necrosis inside the hypertrophied septal wall.

Definition List 1

| Term | Definition |
|---|---|
| HOCM | Hypertrophic Obstructive |
| | Cardiomyopathy |
| myotomy/myectomy | incision/excision of myocardium |
| TASH | transcoronary ablation of septal |
| | hypertrophy |
| LCSH | laser coagulation of septal hypertrophy |

HOCM is almost a familial disease affecting one out of 500 people. Anatomically, HOCM is characterised by an (asymmetrically) hypertrophied inter-ventricular septum causing a dynamic obstruction of the left, and in some patients also of the right outflow tract [1]. The disease is often accompanied by a severe diastolic dysfunction of the left ventricle, deformations/dysfunctions of the mitral valve apparatus, and ischemic heart disease [2].

Major clinical symptoms include exercise-induced breathlessness and syncope, angina, and a high risk of sudden cardiac death [3, 4]. The incidence of sudden death in HOCM is 2%-6% per year [5]. In young patients up to 30 years of age, HOCM is the most frequent cause of sudden cardiac death [6]. Current methods for treatment of symptomatic HOCM patients focus on the reduction or elimination of the intra-ventricular obstruction.

A first well-known treatment of HOCM is drug treatment. According to latest studies, neither negative inotropic drugs, such as beta-blockers and calcium-blockers, nor amiodarone clearly reduced inter-ventricular obstruction of the human heart or incidence of sudden cardiac death [7, 8].

Another well-known treatment of HOCM is cardiac pacing. According to recent studies, atrial or dual chamber pacing with short atrio-ventricular interval can reduce or even abolish the obstruction of the outflow-tract acutely, but long term results were disappointing [9].

A third method of treatment of HOCM is surgical reduction of the hypertrophic inter-ventricular septum, which can be performed by a transaortic myotomy/myectomy. However, perioperative complications include defect of the ventricular septum (1.9%), complete atrio-ventricular conduction block (4.3%), and cerebral embolism (1.1%) especially after relatively often needed re-interventions [10].

A fourth method of reduction of HOCM is known as TASH (Transcoronary Ablation of Septal Hypertrophy). In this method, alcohol is injected via a coronary catheter into the ventricular septum [11]. The injected alcohol produces a chemical infarction known as “transcoronary alcohol ablation” with an increase of creatin-kinase up to 2500 IU/I and is followed by subaortal scarring. However, in some cases atrio-ventricular conduction disturbances can occur with the need for implantation of a permanent pacemaker.

It is therefore an object of the present invention to provide a method for treatment of HOCM which is less detrimental to a patient and easier to apply.

These and other objects of the invention are achieved by a method for treatment of HOCM in which a laser catheter means is introduced into a patient's body, advanced into a heart cavity and directed towards a septal wall of the heart. After positioning of the laser catheter means, laser light is applied via an optical fiber to the septal wall, whereby a coagulation necrosis inside the septal wall is produced. As a result of laser treatment, the hypertrophied muscle is coagulated, healing to a dense fibrous scar which shrinks and cannot contract during systole anymore. Thus, a patient suffering from HOCM can be successfully healed.

With this laser irradiation technique, deep transmural lesions can be produced in diseased septal muscle in an online controllable manner. This method is easy to apply and painless. Procedure duration as well as fluoroscopy times are relatively short, substantially contributing to the patient's comfort and saving costs. Laser lesions can be produced without unwanted effects such as carbonization and tissue vaporization with crater formation (as in [12]) and without damage of coronary vessels (as in [13]). In addition, surgical interventions with the attendant risks, medication with its side effects, and pacemaker implantation can be avoided.

During laser application, the distal tip of the optical fiber is preferably held at a given distance from the myocardium. This allows for a stable and well controllable irradiation process without unwanted thermal damages to the irradiated endo-myocardium.

The laser catheter means of the invention preferably comprises an optical fiber and a probe which is mounted at the distal end of the optical fiber. The distal tip of the optical fiber is preferably fixed at a given distance from the distal end of the device. The probe preferably has a distal cavity in which the light emitted from the optical fiber propagates to the tissue to be treated.

The laser energy applied to the septal tissue is preferably laser light with deep penetration into the myocardium (e.g. wavelengths of 1000 nm-1200 nm,) preferably about 1100 nm, equivalent to a power of about 20 W for a time of up to 60 s. This relatively small amount of energy is sufficient for producing a deep lesion in a hypertrophied ventricular wall. Consequently, application times are relatively short.

According to a preferred embodiment of the invention a bipolar or unipolar local intracardiac electrocardiogram is recorded during laser application and the detected electrical signals of the heart are monitored. Thus, the progress of laser treatment may be followed. Laser application is preferably stopped when the monitored electric signals of the heart fall definitively below a given threshold value. Gradual reduction of the local electrical amplitudes reflect the spread of coagulation-necrosis in the myocardium.

According to a preferred embodiment of the invention the laser catheter is introduced pervenously into the heart. Pervenous access and laser application from the right ventricle is preferred over a retrograde catheterization for reasons of a simpler procedure and is avoiding other detrimental effects on the patients such as arterial puncture, coronary infarction, thrombo-embolic accidents and damages of coronary arteries or of the aortic valve.

Further, the laser catheter is preferably advanced into the right ventricle of the heart and the inter-ventricular septum is irradiated with laser light.

According to a preferred embodiment of the invention the laser catheter is positioned inside a heart cavity using one or more steps of the following method: At first, a guide wire is introduced pervenously and is advanced into a heart cavity. Then, a guiding catheter set, preferably comprising an inner sheath, an overriding outer sheath and a dilator, which is housed inside a lumen of the inner sheath, is advanced over the guide wire into the heart cavity (e.g. the right ventricle). More or less sheaths may be applied as appropriate. At least one of the sheaths is preferably pre-shaped according to the anatomical structures of the targeted region. This contributes to an easy manipulation of the distal catheter tip.

After positioning of the guiding catheter set at the target region, the dilator and guide wire are preferably withdrawn and removed. Thereafter, the laser catheter, preferably comprising an optical fiber and a distal probe, is introduced into the lumen of the inner sheath and advanced to the region to be treated. The distal tip of the laser catheter is preferably advanced beyond the distal end hole of the inner sheath and brought into contact with the pathological region. Thereby, a probe which is mounted at the distal end of the laser catheter is kept in a stable contact with the septal wall. Once a stable contact is achieved, the laser is activated. During continuous saline flow and electrocardiographic monitoring, laser light is applied until the amplitudes of electrical potentials recorded via distal electrodes of the laser catheter are substantially reduced or abolished for good. Prior to removal of the catheter, pressure measurements may be performed in order to confirm that the outflow tract obstruction is abolished.

Numerous advantages and features of the present invention will become readily apparent from the following detailed description of the invention and the embodiments thereof with reference to the accompanying drawings.

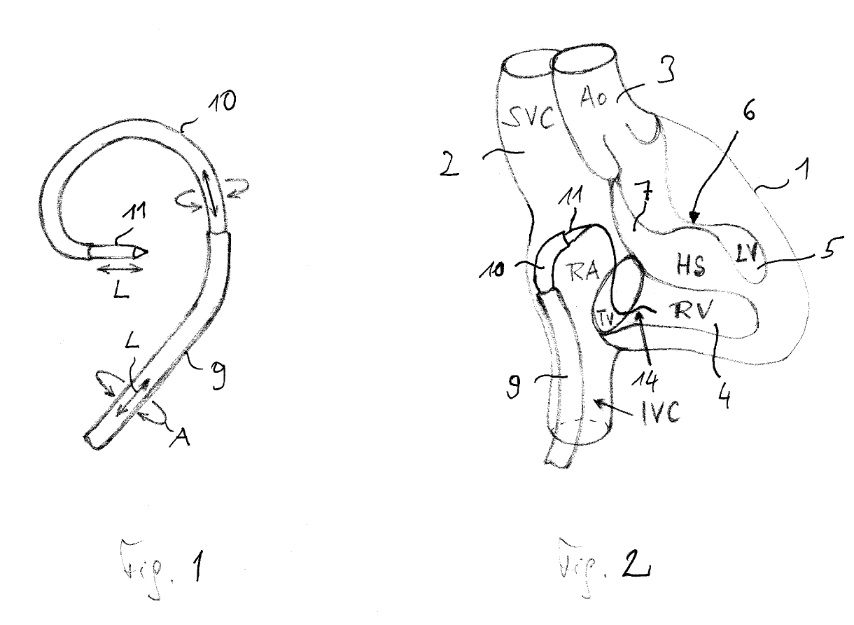

FIG. 1 is a schematic view of an introducer catheter set.

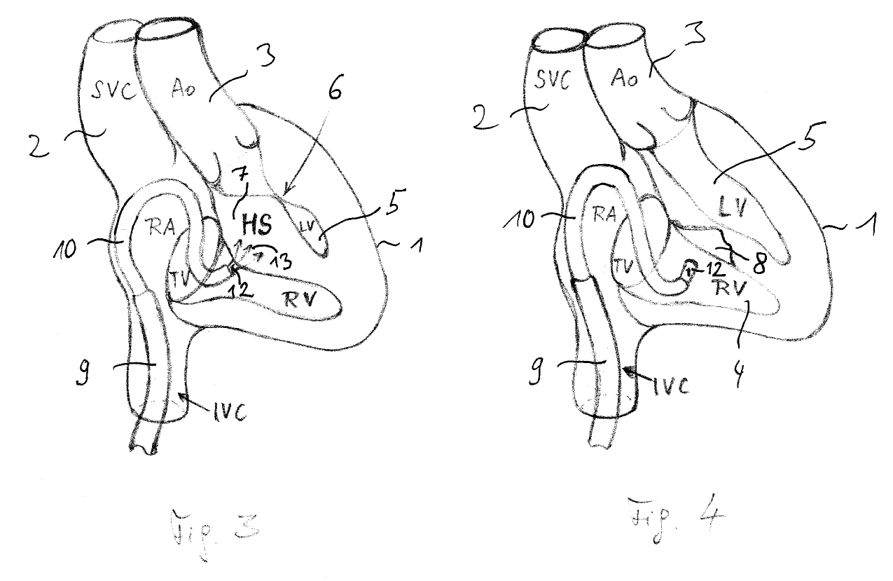

FIG. 2 is a schematic view of a human heart having the catheter set of FIG. 1 introduced into the right ventricle.

FIG. 3 is a schematic view of a human heart with a laser catheter introduced into the right ventricle during laser application.

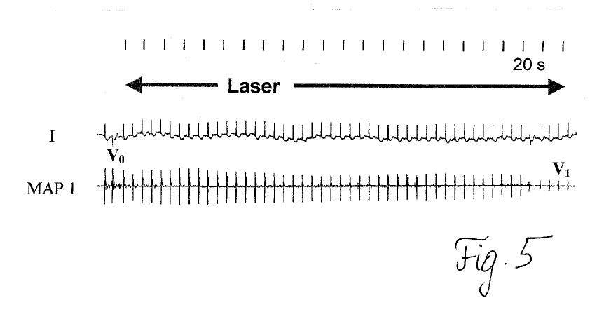

FIG. 4 is a schematic view of a human heart showing the clear-cut lesion of coagulation necrosis produced in the hypertrophied septum after laser application.

FIG. 5 is a surface lead electrocardiogram and intracardiac bipolar mapping electrogram.

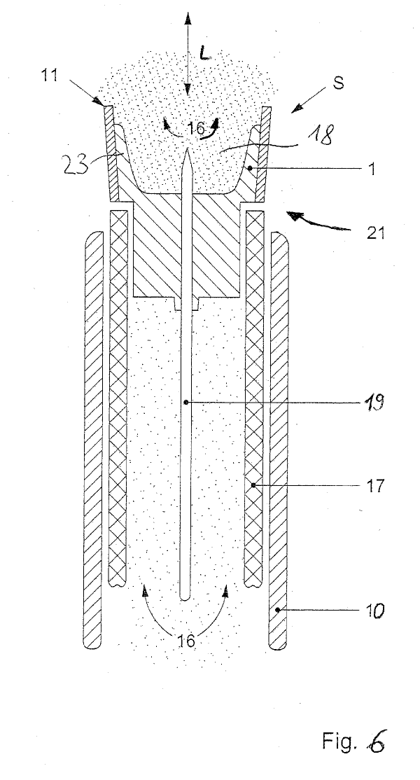

FIG. 6 is a sectional view of a heart catheter with a guiding catheter and a probe movable therein.

FIG. 1 is a schematic view of a distal portion of an introducer/guiding catheter set, comprising a pre-shaped longitudinal outer sheath 9 overriding a pre-shaped longitudinal inner sheath 10 and a dilator 11 which is housed inside a lumen of the inner catheter 10. The individual sheaths 9, 10, 11, can be moved in a longitudinal direction L as well as twisted along a circumferential direction A. The distal portion of the inner catheter 10 as well as the outer catheter 9 have a pre-shaped configuration which is adapted to the anatomical structures of the region to be treated.

In order to manipulate the guiding catheter set 9 and 10 upon the target region, the individual sheaths are advanced and withdrawn relatively to each other in a longitudinal direction L and twisted in a circumferential direction A as appropriate. FIG. 1 shows a position of the guiding catheter set in which the dilator 11 is advanced beyond the end hole of the pre-shaped inner sheath 10 which is in turn advanced beyond the distal end of the overriding outer sheath 9. The dilator 11 is loaded with a guide wire 14 (see FIG. 2) which extends beyond the end hole of the dilator 11. The depicted guiding-introducer set is adapted for targeting septal regions of a heart when inserted percutaneously through a vein from the groin.

Application of the guiding catheter set and introduction of the laser catheter is explained subsequently with reference to FIGS. 2 to 6.

FIG. 2 is a sectional view of a human heart 1 in systole with a severely hypertrophied septum 7 and an almost totally obstructed outflow tract of the left ventricle 5 (LV).

After puncture of a vein in the groin with a needle (Seldinger technique), a guide wire 14 is advanced into the right heart. The needle is removed and the pre-shaped guiding catheter set as shown in FIG. 1, comprising a dilator 11, an inner sheath 10 and an outer sheath 9, is advanced over the guide wire 14 into the right ventricle. The guide wire 14 and dilator 11 are removed, whereupon the resilient sheaths 9, 10 regain their initial configuration with pre-shaped curvatures due to the distinct “memory effect” of the plastic material. The catheter system is now manipulated upon the target region by advancing and withdrawing and by twisting the sheaths 9 and 10. Resilience and flexibility of the sheaths help to keep the distal end of the catheter set in an approximately perpendicular orientation to, and in a stable intimate contact with the moving septal wall 7 in the beating heart 1.

The position of obstruction is marked by an arrow 6. The distal portion of the inner sheath 10 is pointing towards the hypertrophied region. After removal of the guide wire 14 and dilator 11, a laser catheter device 21 (see FIG. 6) is introduced into the lumen of the inner sheath 10 and advanced beyond the tip of the latter. When a probe S (see FIG. 6) of the laser catheter device 21, which is mounted at the distal end of an optical fiber 19, is in contact with the target area, the laser source is activated and the tissue is irradiated at 15-25 W for a time of 30-90 s dependent on the wavelength of the laser light applied, and the relation of power and time of application. The state in which laser light is irradiated on the hypertrophied muscle is shown in FIG. 3.

The laser catheter is rinsed with heparinized (5000 IU/I) saline continuously at a rate of 3-4 ml/min which is increased automatically to 8-15 ml/min during laser application. Laser light is applied under electrocardiographic monitoring until the amplitudes of electrical potentials (see FIG. 5, signal V1) which are recorded via distal electrodes 22 of the laser catheter (see FIG. 6) are substantially reduced or even abolished. Prior to removal of the guiding catheter set 9, 10 and the laser catheter 17, 19, 20 a pressure measurement may be performed in the left ventricle 4 and aorta (or femoral artery). Further, a left ventricular angiography helps to confirm that outflow tract obstruction is abolished.

FIG. 4 is a sectional view of a heart after laser irradiation of the hypertrophied septum 7. The former hypertrophied septal wall 7 now is partially coagulated and substantially thinner, especially during systole, without weakening the septal wall and without aneurysm formation. As can be seen, the obstruction 6 in the outflow tract of the left ventricle 5 is abolished and the patient is cured from HOCM without damage of the anatomic integrity of the septum and without tissue vaporization with crater formation.

FIG. 5 shows some electrical signals of the heart 1 during laser application, namely a surface lead electrocardiogram I and an intracardiac bipolar mapping electrogram (MAP 1) recorded via electrodes 22 of the laser catheter device 21.

FIG. 6 is a sectional view of a laser catheter device 21 (substantially including elements 19, 20) with a guiding catheter 10 (=inner sheath) and a probe S which is movable therein in an axial direction (arrow L). The treatment of HOCM is performed by placing the probe S in an approximately perpendicular position on the target region, and by subsequent laser irradiation of the tissue. The probe S is manipulated via a plastic tube 17 attached to a proximal portion 2 of the probe S. It consists of a probe body 20 made of a single piece, preferably of a plastic material. The probe body 20 has a proximal portion and a distal portion; the proximal portion having a smaller cross-section than the distal portion. Further, the probe body has a central receptacle 24 for receiving the optical fiber 19, the tip of which protrudes into a cavity 18. The cavity 18 is formed in the distal portion of the probe body 20 and surrounded by an (essentially) rigid wall 23. The latter is stable enough to withstand the outer blood pressure without distortion, and it can keep blood out of the cavity 18 to allow for an undisturbed light propagation from the tip of the fiber 19 to the tissue within the cavity 18. The distal portion of the probe body 20 has a distal opening at a location where the probe body 20 contacts the tissue or is at least light-transmissive in that direction.

For treatment of pathological areas, the tip of the optical fiber 19 is fixed inside the probe body 20, thereby keeping the fiber tip at a defined distance from the tissue, on which the probe S is placed. The distal end of the optical fiber 19 may be pointed or flat.

The probe body 20 comprises several electrodes 22 which are placed in recesses on the outer circumference of the distal portion of the probe S, so that the outer surfaces of the electrodes 22 flush with the outer surface of the distal portion of the probe body S. This allows for a smooth advancement of the probe through the haemostatic valve and the guiding catheter 10 (or).

Besides the type of a laser catheter of FIG. 6 any other well-known laser catheter may be used for a treatment of HOCM.

### LITERATURE REFERENCES

1. Schwartz K: Familial Hypertrophic cardiomyopathy: Nonsense versus missense mutations. Circulation 1995; 91:2865-2867.
2. Klues H G, Maron B J, Dollar A L, Roberts W C: Diversity of structural mitral valve alterations in hypertrophic cardiomyopathy. Circulation 1992; 85:1651-1660.
3. Wigle E D, Rakowski H, Kimball B P, Williams W G: Hypertrophic Cardiomyopathy: Clinical spectrum and treatment. Circulation 1995; 921680-1692.
4. Marian A J, Mares Jr. A, Kelly D P, et al: Sudden cardiac death in hypertrophic cardiomyopathy. Eur Heart j 1995; 16:368-376.
5. McKenna W J: Sudden death in hypertrophic cardiomyopathy: assessment of patients at high risk. Circulation 1989; 80:1489-94.
6. Liberthson R R: Sudden death from cardiac causes in children and young adults. New Engl J Med 1996; 334:1039-1044.
7. McKenna W J, Krikler D M, Goodwin J F: Arrhythmias in dilated and hypertrophic cardiomyopathy. Med Clin North Am 1984; 54:802-807.
8. McKenna W J, Harris L, Perez G, et al: Arrhythmia in hypertrophic cardiomyopathy. II. Comparison of amiodarone and verapamil in treatment. Br Heart 1981; 46:173-177.
9. Kappenberger L, Linde C, Daubert C, et al: Pacing for obstructive hypertrophic cardiomyopathy. Eur Heart J 1997; 85:1249-1256.
10. Henric B, Lytle B W, Miller D P, et al: Surgical management of hypertrophic obstructive cardiomyopathy: Early and late results. J Cardiovasc Surg 1995; 110:195-208.
11. Sigwart U: Non-surgical myocardial reduction of hypertrophic obstructive cardiomyopathy. Lancet 1995; 346:211-214.
12. Weber H P, Heinze A, Enders S, et al: Laser catheter coagulation of normal and scarred ventricular myocardium in dogs. Lasers Surg Med 1998; 14:109-119.
13. Weber H P, Enders S, Coppenrath K, et al: Effects of Nd:YAG laser coagulation of myocardium on coronary vessels. Lasers Surg Med 1990; 10:133-139.

### LIST OF REFERENCE NUMERALS OF THE DRAWINGS

1 Human heart
2 Right atrium
3 Aorta
4 Right ventricle
5 Left ventricle
6 Obstruction
7 Intra-ventricular septum
8 Lesion
9 Outer sheath
10 Inner sheath
11 Dilator
12 Laser catheter
13 Laser light
14 Guide wire
16 Saline solution
17 Tubular hose
18 Application cavity
19 Optical fiber
20 Probe body
21 Laser catheter
22 Electrodes
23 Circumferential wall
24 Receptacle
L Longitudinal direction
A circumferential direction

What is claimed is:

1. A method for treatment of hypertrophic obstructive cardiomyopathy (HOCM) in a heart, particularly a human heart, wherein a laser catheter means is introduced into a patient's body, advanced into a heart cavity and directed towards a septal wall of the heart, characterized in that laser light is applied to the septal wall so as to produce a coagulation necrosis inside the hypertrophied septal wall.

2. The method of claim 1, wherein laser light with appropriate deep penetration into the myocardium, having a wavelength of about 1100 nm.

3. The method of claim 1, wherein laser light with and a power of 15-25 W and a time of 30 s-90 s per application is applied to the septal wall.

4. The method of claim 1, wherein an electrocardiogram is recorded and local intracardiac electrical signals of the heart are monitored.

5. The method of claim 4, wherein the application of laser light is stopped, when the electric signals of the heart permanently fall below a given value.

6. The method of claim 1, wherein the laser catheter means is introduced pervenously into the heart.

7. The method of claim 1, wherein a guiding catheter set, comprising an inner sheath, an overriding outer sheath and a dilator, which is housed inside the inner sheath, is advanced over the guide wire into the heart.

8. The method of claim 1, wherein a laser catheter means comprises an optical fiber which is connectable to a laser energy source and has its tip mounted in a lumen of a distal probe.
